(19) United States
(12) Patent Application Publication
(10) Pub. No.: US 2021/0338370 A1
(43) Pub. Date: Nov. 4, 2021

(54) Head Strap for Medical Loupes

(71) Applicants: Connor Lewis Bryant, Ottershaw (GB); Priyam Timir Patel, Cheam (GB)

(72) Inventors: Connor Lewis Bryant, Ottershaw (GB); Priyam Timir Patel, Cheam (GB)

(73) Assignee: Bryant Medical Ltd, Chertsey (GB)

(21) Appl. No.: 16/864,167

(22) Filed: May 1, 2020

(51) Int. Cl. A61B 90/50; G02C 3/00

(52) U.S. Cl. CPC A61B 90/50; A61B 2090/3616; G02C 3/003

(57) ABSTRACT

Disclosed is a method and system to construct a head strap for a dental or medical pair of magnifying loupe glasses. Key features of the disclosure are that the head strap is hygienic and very easy to disinfect, while at the same time being easy and quick to adjust and securely attach to the head, and quickly and securely attach to the temples of a standard loupe.

### FIELD OF THE INVENTION

The present invention relates broadly to vision aids, and more specifically to retaining corrective lenses to the head of a person.

### BACKGROUND OF THE INVENTION

Loupes are small magnification devices used to see small details more closely. As dental professionals use both hands in performing dental procedures, dental loupes are binocular and usually take the form of a pair of glasses.

Loupes are worn by dentists not only to increase the details that can be seen, but also to improve posture by avoiding slouching to view inside a mouth. Many dentists mount a headlight, with an associated battery pack, on the loupes to increase visibility.

Loupes are supplied ‘fitted’ to the dentist's face and can be supplied with prescription lenses for vision correction. Loupes are also common in other professional sectors such as surgery and single eyeglass loupes in professional sectors such as jewellery, geology and printing.

Many dentists use common eyewear head straps, also known as retainers, lanyards or straps, attached to the temples (the arms) of the loupe frames. Dentists attach head straps to prevent the loupes from falling and becoming damaged as well as, in the case of head straps with an adjustable length, securing the loupes more tightly on the face to prevent them slipping down the nose bridge when looking down or becoming unsettled as the dentists moved his head up or down or rotated left/right.

The risk of loupes slipping off a face is much greater than that of wearing normal eyewear as the loupes are heavier because of the magnification lenses and optional headlight, and also because the loupes are unbalanced and have a heavier weight load on the nose pads due to the mounting of the lens and headlight compared to the temples.

Technology is progressing to reduce the weight of loupes, however at the same time technology advancements mean that higher magnification loupes are becoming available with longer and heavier lenses.

In 1942 Corcoran (U.S. Pat. No. 2,270,931) disclosed a method of supporting heavy optical instruments over eyes. By holding the heavy optical instruments using a pair of parallel bars, which are hinge mounted to head gear comprising two or three fixed length, elongated straps pivotally attached to each other and mounted over the head the weight of the instruments is spread over the head. This arrangement also provides a hands-free method to ensure the optical instruments are always positioned over the eyes pointing forwards as the head moves.

Later in 1955 Schmidt (U.S. Pat. No. 2,935,910) disclosed a method to construct a headband loupe for doctors and other workers in which the lenses, lens holders and other parts of the loupe are made removable and interchangeable to change the level of magnification and also accommodate people wearing glasses behind the lenses. The loupe is secured using rivets to an elasticated headband fastened with buckles or snaps around the circumference of the head. The use of buckles or snaps allows the headband to be adjusted to ensure a tight fit, and the use of an adjustable bolt between the headband and the lens holders allows for the loupe to be tilted upwards away from the eyes when not required.

Price (U.S. Pat. No. 4,234,910) in 1978 disclosed a method to construct a head supported illumination device consisting of the mounting of fibre optics from a light generating machine onto the temples of existing glasses for physicians and surgeons. To secure the frame of the glasses, an adjustable elasticated strap is used to secure them to the back of the head. The strap is attached to the temples using a rubber loop attached to the end of the temple and is adjusted using a buckle.

Bastable and Goldberg (U.S. Pat. No. 4,807,987) in 1986 disclosed a method to mount an interchangeable set of loupe lens together with a fibre optic light source for ear, nose and throat surgery. The mounting is attached to an upholstered, adjustable headband around the circumference of the head together with an additional strap over the top of the head to allow comfortable use for a number of hours and allow adjustments for individual head sizes using fasteners as well as brackets to position the viewing system and light system in front of the user's eyes.

Later in 1994 Sherlock (U.S. Pat. No. 5,548,841) disclosed a method to construct a headband for a loupe using a one-piece polypropylene headband that provides an easy method to quickly slip the loupes on and off the head, together with a compression force that provides a method to secure the loupe for all head sizes.

Qingfeng (China Patent CN2215728Y) in 1994 disclosed a method to mount magnification lenses with variable magnification on a frame attached to the head using an adjustable rubber belt. This has the benefit of being smaller than traditional loupes and allowing variable magnification. The rubber belt is simple to adjust.

Donny's application in 2019 (WIPO Patent Application PCT/US2019013777) disclosed a method to build a head strap for a standard pair of loupes available for sale from various manufacturers using two monofilaments which are attached to the temples of the loupes using an elasticated coupler. The monofilaments pass through a tight sleeve near each of the temple, with a stopper made of rubber or plastic on the other end. The sleeve allows for size adjustment and the monofilament loop from each temple passes over the back of the head. The method provides an adjustable head strap for existing loupes which is very light weight and displaces the weight of the loupes to the back of the head. The use of monofilaments makes the loop more hygienic and easier to clean.

While these examples provide methods to construct a loupe and headband combinations and also methods to manufacture a headband to attach a standard pair of loupes to a head to displace the weight of the loupes over the head, and to securely attach the magnification lenses so that they remain stable as the head moves, what makes the method in the present application unique from Corcoran, Schmidt, Price, Bastable, Sherlock, Qingfeng and Donny is a method of constructing a head strap for a standard paid of loupes which is hygienic and very easy to disinfect, whilst at the same time being easy and quick to adjust and securely attach to the head, and quickly and securely attach to the temples of a standard loupe. There are no parts which can catch hair or be caught on clothes and the whole surface of the head strap can be simply disinfected using wipes. There is no requirement to adjust for size every time the loupe is placed on the head and the one size head strap is constructed to fit most of the dental and surgical standard loupes available for sale.

### SUMMARY OF THE INVENTION

The present invention enables a dentist or surgeon to quickly and securely attach a set of loupes to their head whilst ensuring that the loupe head strap is hygienic and very easy to clean, is easy to adjust its size to ensure a tight fit whilst also being easy and quick to attach and detach. The method also ensures that one size of head strap is able to fit many different manufacturers loupes using a safe and hygienic method of attaching them to the loupes.

In one embodiment the dentist or surgeon attaches a head strap made from pieces of silicone tube to the loupes by simply sliding the ends of the two hanging ends over the ends of the loupe temples. The size, and the texture of the inside surface of the silicone ensures a tight fit on most standard loupes whilst the smooth outer surface ensures a highly hygienic surface which is easy to disinfect with wipes. The size of the head band is adjusted by sliding cylindrical magnets located inside the silicone tubes up, or down. Once the loupes are placed on the head, a simple hand movement to touch the two magnets together secures the head strap to the lower back of the head and ensures that the loupes are steady, as well as transferring the weight of the loupes away from the nose bridge to the back of the head. The weight of the connector at the end of the head strap behind the head can be adjusted to balance the weight of the loupes front to back. In this embodiment the head strap is easily disinfected, is light weight, is easily adjusted, fits most manufactured loupes and is simple and quick to attach and detach from the head.

The present invention attaches to the loupes with a simple hand motion and also simply adjusts the size of the head strap while ensuring that the whole assembly is hygienic and easy to disinfect, and can be securely attached to most of the manufactured loupes available today.

Many features and advantages of the present invention will become apparent from reading the following description, when considered in conjunction with the accompanying drawings.

### BRIEF DESCRIPTION OF THE DRAWINGS

FIG. 1 is a side elevation view of a person wearing a pair of loupes with a head strap, in accordance with one embodiment of the present invention.

FIG. 2 is a perspective view of a person wearing a pair of loupes with a head strap in accordance with one embodiment of the present invention.

FIG. 3 is a perspective view of the head strap with the magnets not connected.

FIG. 4 is a perspective view of the head strap with the magnets connected.

FIG. 5 is a perspective view of the end of the silicone tube of the head strap that is pushed over the temples of the loupes.

### DETAILED DESCRIPTION

In the preferred embodiment the dentist or surgeon disinfects their head strap by wiping with a disinfect wipe. The user then attaches the head strap to a pair of loupes which the dentist of surgeon owns, purchased from one of the many manufacturers selling such items. The user then adjusts the fitting of the head strap by moving the magnets inside the head strap silicone tubes. Once adjusted the user places the loupes on the head, with the loupes resting on the bridge of the nose and the loupe temples resting over the ears of the user. The head strap is passed over the head and using a simple hand motion the head strap is tightened by pushing the magnets together. The user is then free, with two hands, to continue with his work.

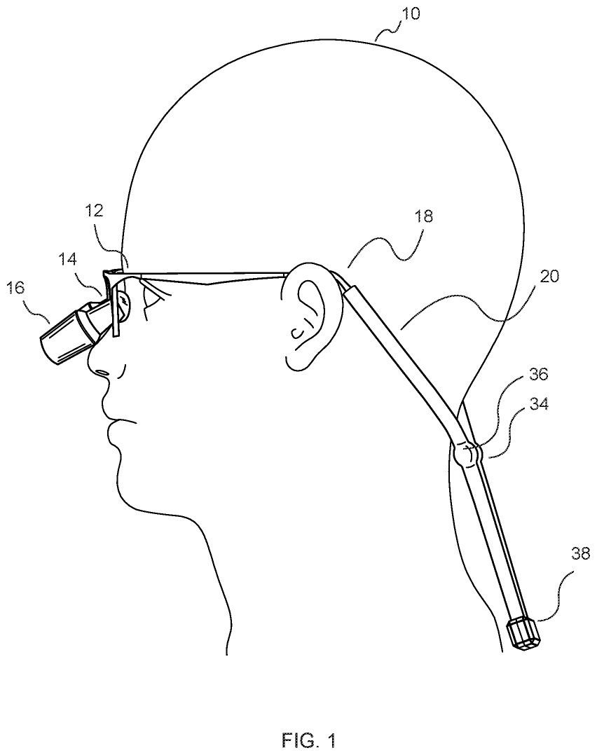

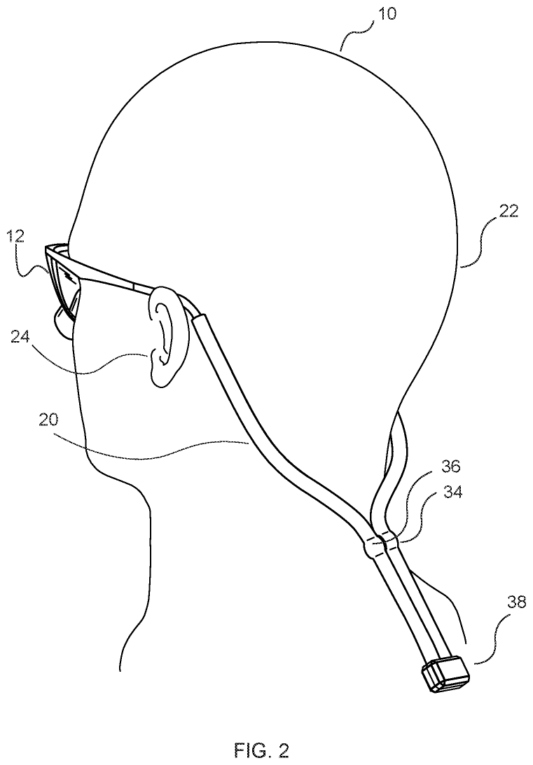

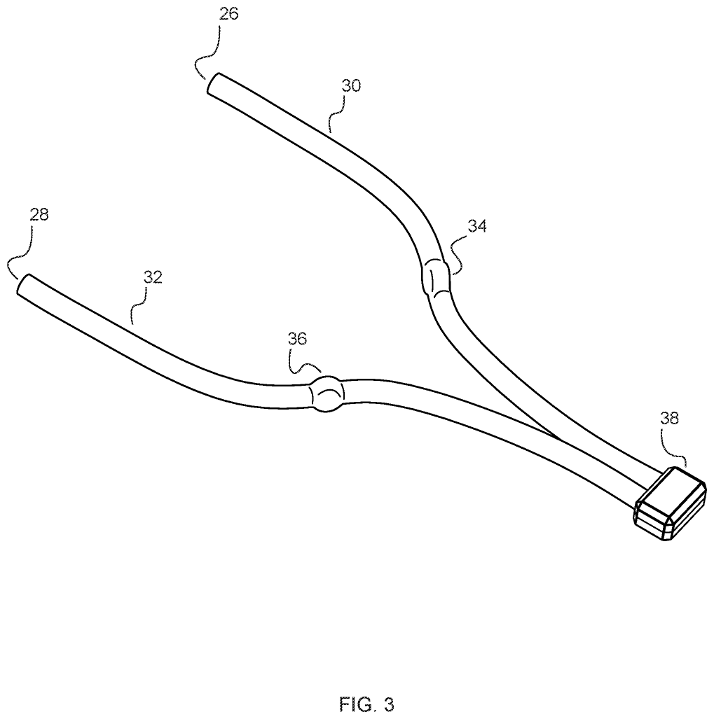

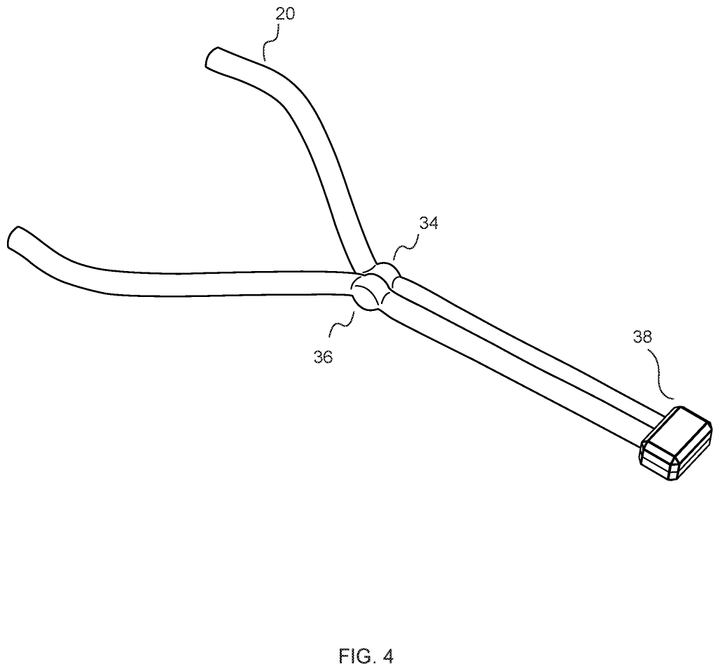

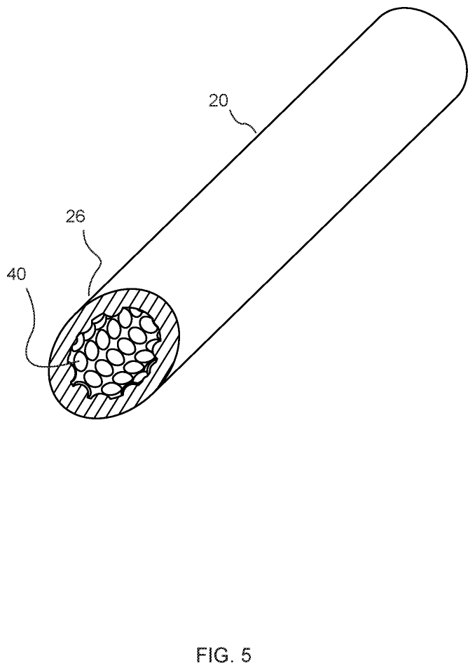

Reference is made to FIG. 1 which shows a side elevation view of a person wearing a pair of loupes with a head strap, FIG. 2 which shows a perspective view of the same person wearing the pair of loupes and head strap, FIG. 3 and FIG. 4. which show perspective views of the head strap on its own and FIG. 5 which shows a perspective view of the end of the silicone tube of the head strap which is attached to the temples of the loupes.

In step 1, the dentist or surgeon disinfects the head strap 20 by wiping the outer smooth silicone surface 30 and 32 and aluminum connector block 38 with a readily available disinfectant wipe, or by boiling the head strap 20 in water for 3 to 4 minutes. The head strap is easy to clean because the uniform construction of the head strap means there are no sharp edges to catch either the wipe or other foreign objects such as hair and also because the head strap is made from silicone material. Silicone is quick-drying, non-porous and hypoallergenic and is 35 times more hygienic than nylon and therefore the head strap is highly more hygienic than a pair of nylon or cotton retainers.

In step 2, if this is the first time that the user is wearing the head strap they will adjust the size of the head strap by moving the Neodymium N52 magnets 34 and 36 located inside the silicone tubes up or down by a gentle squeeze to ensure the distance between the ends of the head strap 26 and 28 and the magnets 36 and 38 is approximately the distance between the ears 22 and 24 and the back of the head.

The disc shaped magnets 36 and 38 have been designed with dimensions of 8 mm diameter and 5 mm width, which sit inside the silicon tubing 30 and 32 which has an internal diameter of 4 mm. This combination of sizes has been found to be optimal to allow the magnets to move without slipping. The magnets 34 and 36 have flat faces to ensure there are no ‘pinch points’ which can wear excessively when the magnets are constantly connected and reconnected.

In step 3, the user attaches the ends of the head strap 26 and 28 to the loupes 12 by inserting the ends of the temples 18 into the ends of the head strap 26 and 28 using a push and twisting motion until tight. The inside of the ends of the silicone tubing 26 and 28 have been designed with a textured internal surface 40 so that the silicone both grips more to the surface of the temples arms using the raised surface elements and also at the same time allows for the silicone material to stretch between the raised surface elements to accommodate as many different sizes and styles of loupe temple arms as possible.

Once the head strap 20 is attached to the loupes 12, in step 4, the user guides the temples of the loupes 18 either side of the head 10 and over the ears 22 and 24 and the nose bridge of the loupes 14 sit on the user's nose Whilst guiding the loupes the user ensures that the head strap 20 is placed over the head 10.

In step 5, once the loupes are comfortable on the ears 22 and 24 and nose 14, the user grasps the magnets 34 and 36 and pushes them together behind the head until the magnets come together due to their magnetic attraction. If the head strap is found to be too loose or tight on their first use, the user can pull the magnets 34 and 36 apart and slide them up or down further inside the silicone tube 30 and 32 as required to re-adjust the size and then ‘click’ them together again.

During the dental or surgical procedure, the user can view in detail the area to be examined using the loupe magnification lenses 16. If the user needs to move their head from side to side, or up and down, the loupe strap 20 ensures that the loupes 12 remain stable on the nose and ears 22 and 24 and do not slip so much that the view down the magnification lenses 16 is not straight or distorted, or that the loupes 12 do not fall due to their weight and unbalanced nature and potentially break.

In an alternative embodiment, where heavier or larger loupes 12 are required, the aluminum connector block 38 can be replaced with an alternative heavier weighted block to counterbalance the weight of the loupes on the nose, and over the ears 22 and 24 to ensure a more stable sitting of the loupes 12 on the head 10.

Once the user has completed their dental or surgical procedure, the user can pull the magnets 34 and 36 apart to loosen the loupes, and the loupes can be taken on the face in a forward motion, with the head strap 20 passing over the head 10.

What is claimed is:

1. A head strap for medical loupe glasses, comprising:
a pair of tubes of flexible material joined together at one end of each tube; and
magnets physically attached to each tube at points along its length,
wherein said tubes are attachable to temples of a pair of eyeglasses and said magnets are joined together by magnetic attraction.

2. The head strap of claim 1 wherein said flexible material is silicone.

3. The head strap of claim 1 wherein said flexible material is rubber.

4. The head strap of claim 1 wherein said flexible material is polyvinyl chloride.

5. The head strap of claim 1 wherein said flexible material is polyurethane.

6. The head strap of claim 1 wherein said flexible material is nylon.

7. The head strap of claim 1 wherein said flexible material is cotton.

8. The head strap of claim 1 wherein said flexible material is braided fiberglass.

9. The head strap of claim 1 wherein said flexible material is fluoropolymer.

10. The head strap of claim 1 wherein said tubes are connected by a weighted block selected from a plurality of blocks having different weights.

11. The head strap of claim 1 wherein said tube has a rough inner surface.

12. A head strap for medical loupe glasses, comprising:
a pair of tubes of flexible material joined together at one end of each tube; and
magnets inserted in each tube wherein the magnets are free to move within the tubes,
wherein said tubes are attachable to temples of a pair of eyeglasses and said magnets are joined together by magnetic attraction.

13. The head strap of claim 12 wherein said flexible material is silicone.

14. The head strap of claim 12 wherein said flexible material is rubber.

15. The head strap of claim 12 wherein said flexible material is polyvinyl chloride.

16. The head strap of claim 12 wherein said flexible material is polyurethane.

17. The head strap of claim 12 wherein said flexible material is nylon.

18. The head strap of claim 12 wherein said flexible material is cotton.

19. The head strap of claim 12 wherein said flexible material is braided fiberglass.

20. The head strap of claim 12 wherein said flexible material is fluoropolymer.

21. The head strap of claim 12 wherein said tubes are connected by a weighted block, the block selected from a plurality of blocks having different weights.

22. The head strap of claim 12 wherein said tube has a rough inner surface.
